(19) United States
(12) Patent Application Publication
(10) Pub. No.: US 2026/0151636 A1
(43) Pub. Date: Jun. 4, 2026

(54) IMPLANTABLE MEDICAL DEVICE ANTENNA

(71) Applicant: Medtronic, Inc., Minneapolis, MN (US)

(72) Inventors: Yanzhu Zhao, Blaine, MN (US); David J. Peichel, Minneapolis, MN (US); Andrew J. Ries, Lino Lakes, MN (US)

(21) Appl. No.: 19/124,993

(22) PCT Filed: Oct. 6, 2023
(86) PCT No.: PCT/US2023/076217

§ 371 Date: Apr. 28, 2025

Related U.S. Application Data
(60) Provisional application No. 63/381,366, filed on Oct. 28, 2022.

(51) Int. Cl. A61N 1/372; A61N 1/05; A61N 1/365

(52) U.S. Cl. CPC A61N 1/37264; A61N 1/056; A61N 1/365; A61N 1/37223

(57) ABSTRACT

An implantable medical device (IMD) comprises a housing, a communication circuit within the housing, and an antenna. The antenna comprises a forming part of the housing, a first metal portion adjacent to the non-conductive component on a first side of the non-conductive component, a second metal portion adjacent to the non-conductive component on a second side of the non-conductive component and a plurality of switchable contacts configured to couple the dielectric antenna to the communication circuit, wherein the plurality of switchable contacts are configurable according to a. selected excitation mode of a plurality of excitation modes to connect the communication circuit to the antenna via a selected one or more of the plurality of contacts to excite the antenna to provide radiofrequency (RF) wireless communications between the IMD and an external device.

This application is an international application with provisional priority of U.S. Provisional Ser. No. 63/381,366, filed 28 Oct. 2022, the entire contents of which are incorporated herein by reference.

### TECHNICAL FIELD

The disclosure relates to medical device communication and, more particularly, to an antenna for an implantable medical device (IMD).

### BACKGROUND

Various IMDs have been clinically implanted or proposed for therapeutically treating and/or monitoring one or more physiological conditions of a patient. Such devices may be adapted to monitor or treat conditions or functions such as those relating to heart, muscle, nerve, brain, stomach, endocrine organs, or other organs, and their related functions. Advances in design and manufacture of miniaturized electronic and sensing devices have enabled development of IMDs capable of therapeutic as well as diagnostic functions such as pacemakers, cardioverters, defibrillators, biochemical sensors, implantable loop recorders, and pressure sensors, among others. Such devices may be associated with leads that position electrodes or sensors at a desired location, or such devices may be leadless with electrodes or sensors integrated into the device housing. These devices may have the ability to wirelessly communicate with another device implanted in the patient and/or to another device located externally of the patient.

Although implantation of some devices requires a surgical procedure, other devices may be small enough to be delivered and placed at an intended implant location in a minimally invasive manner, such as by a percutaneous delivery catheter or transvenously. By way of illustrative example, implantable monitors have been proposed and used to monitor heart rate and rhythm, as well as other physiological parameters, such as patient posture and activity level. Such direct in vivo measurement of physiological parameters may provide significant information to clinicians to facilitate diagnostic and therapeutic decisions. In addition, miniaturized pacemakers that may be implanted directly within a patient's heart, with or without leads to position electrodes, have been built and adapted to provide pacing and other electrical therapy to the patient.

For most of these devices, the ability to communicate using wireless communication with the device after its implantation is important. These example devices need to communicate with external devices and/or other devices implanted within the patient. For example, the devices may transmit information indicative of the sensed data and/or treatment data. The devices may receive information such as treatment and sensing parameters and other information that may define modes of operation.

### SUMMARY

The disclosure describes an antenna integrated with a housing of an IMD, wherein the antenna may be used to provide communications between the IMD and one or more other devices. In some examples, IMDs that may advantageously include an antenna according to this disclosure may be small devices and may have been implanted within the patient under the skin or relatively deeper within the patient, for example implanted on or within the heart of a patient. An example of such an IMD is the Medtronic® Micra® self-contained pacemaker that is designed to be implanted internally within the heart of a patient and in some cases requires no external leads coupled to the device in order to provide pacing to the heart.

One solution to extend the mission lifespan of an IMD is to provide a larger power source, such as a larger size battery, in the device prior to implantation. However, a larger power source may require an increase in the overall size of the implantable medical device. Due to the need to miniaturize implantable medical devices so that they may be implanted in desired locations, such as within the heart, while maintaining a small size in order to minimize any obstruction, e.g., to blood flow, created by the device once implanted, an increase in the size of the power source, and thus the overall size of the implantable medical device, may be counter-productive for many applications.

The IMD described in this disclosure includes an antenna including a non-conductive component between first and second metal portions formed as part of the housing of the device, which provides a required level of telemetry and communication functionality for the IMD and frees up valuable real estate otherwise allocated for a conventional microstrip or patch antenna. This provides advantages because a same size device may be configured to include a larger battery or an IMD may have an overall smaller size with a similar size battery mission lifespan compared to a device having a conventional microstrip or patch antenna. A longer mission lifespan and/or a smaller overall device size provides benefits to both the patient and the clinicians who implant and/or treat the patient following the implantation of the devices. For example, a longer lifespan may increase the time between when a device is implanted and when a replacement device is needed, and thus increases the time and/or may eliminate the need for the additional surgical process required to implant a replacement device. The miniaturization of the implantable medical device may allow for a less invasive implant procedure by the physician, such as implantation by use of a percutaneous delivery catheter or transvenously, and a smaller implant volume required within the patient.

In addition, some IMDs are implanted more deeply within a patient such that signal strength is a consideration for reliable communication. Also, some types of IMDs may be more difficult to orient within a patient, such that a known orientation of an antenna of the IMD within a patient may be difficult to achieve. In some examples, the antenna, e.g., the non-conductive component, may be ring-shaped, and the IMD antenna as described herein may be able to provide approximately uniform signal strength along multiple axes for RF communication signals between the IMD and other devices, and/or may be configured to adaptively or selectively direct RF communication signals in different directions, to achieve reliable communication regardless of the orientation of the IMD within a patient.

In one example, an implantable medical device (IMD) comprises a housing, a communication circuit within the housing, and an antenna comprising a non-conductive component forming part of the housing, a first metal portion of the housing adjacent to the non-conductive component on a first side of the non-conductive component, a second metal portion of the housing adjacent to the non-conductive component on a second side of the non-conductive component, and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

In another example, pacemaker comprises a housing configured for implantation within a heart chamber of a patient, a plurality of electrodes integrated into the housing, a sensing circuit within the housing, the sensing circuit configured to sense a cardiac electrogram via the one or more electrodes, a therapy delivery circuit within the housing, the therapy delivery circuit configured to deliver pacing pulses via the one or more electrodes, a communication circuit within the housing, and an antenna. The antenna comprises a non-conductive component forming part of the housing, a first metal portion of the housing adjacent to the non-conductive component on a first side of the non-conductive component, a second metal portion of the housing adjacent to the non-conductive component on a second side of the non-conductive component, and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

In another example, a method comprises configuring, by processing circuitry within a housing of an implantable medical device (IMD), a plurality of switchable contacts of an antenna of the IMD according to a selected excitation mode of a plurality of excitation modes, and exciting, by a communication circuit within the housing of the IMD, the antenna via the plurality of switchable contacts. The antenna comprises a non-conductive component forming part of the housing, a first metal portion of the housing adjacent to the non-conductive component on a first side of the non-conductive component, and a second metal portion of the housing adjacent to the non-conductive component on a second side of the non-conductive component. The method further comprises communicating, by the IMD and via radiofrequency (RF) wireless communications, with an external device using the excited antenna.

In another example, an implantable medical device (IMD) comprises, a housing, a communication circuit within the housing, and an antenna. The antenna comprises a ring-shaped non-conductive component forming part of the housing, a first ring-shaped metal portion of the housing adjacent to the non-conductive component on a first side of the non-conductive component, a second ring-shaped metal portion of the housing adjacent to the non-conductive component on a second side of the non-conductive component, wherein the first and second ring-shaped metal portions have a common longitudinal axis with each other and the ring-shaped non-conductive component, and a plurality of switchable contacts distributed circumferentially around the IMD and configured to connect the first and second ring-shaped metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE FIGURES

FIG. 1 is a conceptual drawing illustrating an example medical device system in conjunction with a patient according to various examples described in this disclosure.

FIG. 2A is a diagram of an example IMD with an antenna in accordance with one or more techniques described in this disclosure.

FIG. 2B is a diagram illustrating a portion of the example IMD of FIG. 2A in greater detail.

FIG. 2C is a cross-sectional diagram of the IMD of FIG. 2A taken along line C-C.

FIG. 3A is a diagram of another example IMD with an antenna in accordance with one or more techniques described in this disclosure.

FIG. 3B is a conceptual diagram of features of the example IMD of FIG. 3A in accordance with one or more techniques described in this disclosure.

FIG. 3C is a functional block diagram illustrating an example configuration of the IMD of FIGS. 3A and 3B in accordance with one or more techniques described in this disclosure.

FIG. 4 is a conceptual diagram illustrating an example of antenna radiation with an example antenna operated in two complementary excitation modes in accordance with one or more techniques described in this disclosure.

FIG. 5 is a diagram illustrating antenna gain in an azimuth plane for an example antenna, wherein the antenna is operated in a single directional excitation mode and a dual directional excitation mode in accordance with techniques described in this disclosure.

FIG. 6 is a diagram illustrating antenna gain in an altitude plane for an example antenna, wherein the antenna is operated in a single directional excitation mode and a dual directional excitation mode in accordance with techniques described in this disclosure.

FIG. 7 is a flow diagram illustrating an example operation of an IMD including an antenna in accordance with one or more techniques described in this disclosure.

FIGS. 8A and 8B are perspective and cross-sectional diagrams, respectively, illustrating an example antenna an interconnect device of an IMD in accordance with techniques described in this disclosure.

FIGS. 9A and 9B are cross-sectional and perspective diagrams, respectively, illustrating another example antenna and another example interconnect device of an IMD in accordance with techniques described in this disclosure.

FIG. 10 is a cross-section diagram illustrating another example antenna and another example interconnect device of an IMD in accordance with techniques described in this disclosure.

### DETAILED DESCRIPTION

This disclosure generally relates to an antenna that is formed as part of the housing of an IMD. The antenna may include a plurality of switchable contacts to metal portions adjacent to a non-conductive component for coupling the antenna to a communication circuit of the IMD, which may include switches and/or signal feeds to selectively connect the contacts to the communication circuit. The antenna is configured to be excited to provide RF wireless communications, such as Bluetooth Low Energy signals, between the IMD and one or more other devices, such as an external device or another IMD.

The example non-conductive components may be formed as part of the housing of the medical device such that when the IMD is implanted, the non-conductive component is in contact with patient fluid and/or tissue. Accordingly, this disclosure describes examples of antennas that can be formed as part of the housing of the medical device such that the antennas utilize relatively little or no volume within the housing. This may result in an overall smaller medical device, as compared to other medical devices having a header that houses the antenna, which is beneficial for implantation. This may also result in a medical device having a similar size as prior devices, but which may include more room for a larger power source or other components.

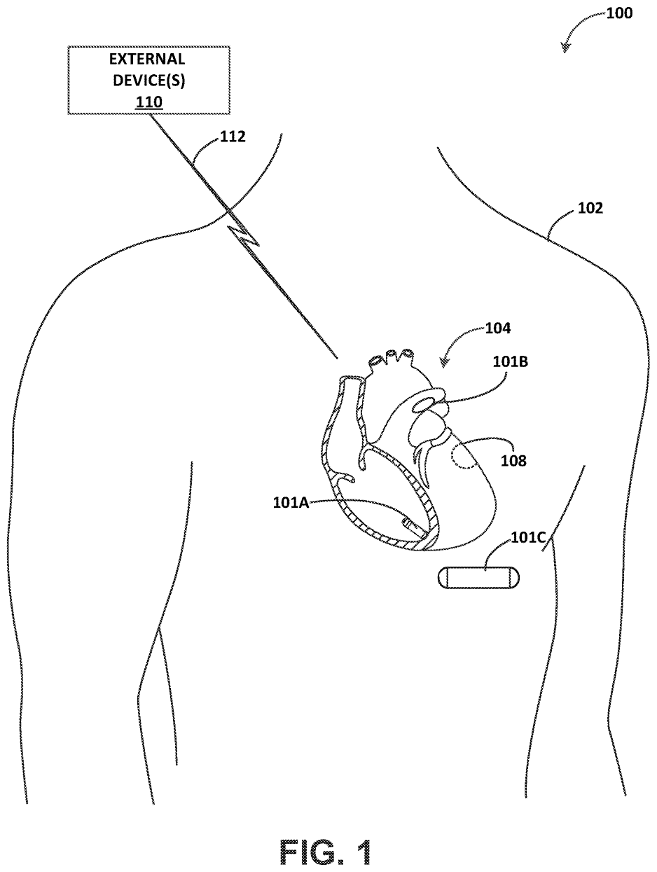

FIG. 1 is a conceptual drawing illustrating an example of some components of a medical device system 100 in conjunction with a patient 102 according to various examples described in this disclosure. The systems, devices, and techniques described in this disclosure provide IMDs that include an antenna arranged in a manner further described throughout this disclosure, to communicatively link the IMD(s) with one or more external devices 110, and/or to each other, as further described below.

System 100 may include one or more IMDs, such as one or more of IMD 101A, IMD 101B, and IMD 101C (collectively, “IMDs 101”), implanted in patient 102. In various examples, at least one of the IMDs 101 in system 100 includes an antenna configured as described in this disclosure. Although the example techniques are described with respect to devices for monitoring the heart and/or delivering treatment to the heart and, in some cases, devices implanted within the heart, the example techniques are not so limited. For instance, the example techniques described in this disclosure may be extended to non-cardiac medical devices (e.g., devices for pain stimulation, brain stimulation, pelvic stimulation, spinal stimulation, etc. and devices such as implanted drug pumps, and the like) that provide wireless communication with other devices. Furthermore, although system 100 illustrates IMDs 101A, 101B, and 101C implanted together in patient 102, systems need not include each of these IMDs 101, and may include any one or more IMDs 101.

System 100 may include an intracardiac pacing device IMD 101A. In the illustrated example, IMD 101A is implanted in the right-ventricle of patient 102, e.g., internal to the heart 104 of patient 102. In some examples, one or more IMDs (not specifically shown in FIG. 1) similar to IMD 101A may additionally or alternatively be implanted within other chambers of heart 104 or attached to the heart epicardially. IMD 101A may be configured to sense electrical activity of heart 104, and/or to deliver stimulation therapy such as pacing therapy, e.g., bradycardia pacing therapy, cardiac resynchronization therapy (CRT), anti-tachycardia pacing (ATP) therapy, and/or post-shock pacing, to heart 104.

IMD 101A may be attached to an interior wall 108 of heart 104 via one or more fixation mechanisms that penetrate the tissue. As described herein, fixation mechanisms may secure IMD 101A to the cardiac tissue and retain one or more electrodes (e.g., a cathode or an anode) on the housing of IMD 101A in contact with the cardiac tissue. In addition to delivering pacing pulses, IMD 101A may be capable of sensing electrical signals using the electrodes carried on the housing of IMD 101A. These electrical signals may be electrical signals generated by cardiac muscle and indicative of depolarizations and repolarizations of heart 104 at various times during the cardiac cycle. In various examples, IMD 101A is configured to wirelessly communicate with one or more external devices 110 as illustratively shown in FIG. 1 by communication link 112.

System 100 may include IMD 101B that may be implanted in various locations of patient 102 outside the ventricles of heart 104 of patient 102. IMD 101B may comprise an implantable pressure sensing device that may be implanted within a left or right pulmonary artery of the patient. IMD 101B may include pressure sensing circuitry configured to measure the cardiovascular pressure within the pulmonary artery of patient 102. In some examples, IMD 101B may include a wireless communication circuit, e.g., TCC and/or RF telemetry circuitry, configured to receive a trigger signal from external device(s) 110, IMD 101C and/or IMD 101A, at electrodes or an antenna provided in IMD 101B (e.g., an antenna such as one of the examples described in this disclosure). The pressure sensing circuitry of IMD 101B may be configured to measure the cardiovascular pressure of patient 102 in response to receiving the trigger signal. In either case, IMD 101B may be configured to transmit the measured pressure values to external device(s) 110, IMD 101C and/or IMD 101A by wireless communication. For example, IMD 101B may transmit measurements and data acquired by IMD 101B related to pulmonary artery pressure and other information generated by IMD 101B to IMD 101C, to IMD 101A, and/or to external device(s) 110. In various examples, IMD 101B comprises an antenna used for communications between IMD 101B and other devices of system 100, arranged using the examples of antennas described throughout this disclosure, or any equivalents thereof.

As illustrated in FIG. 1, system 100 includes IMD 101C which may be an insertable cardiac monitor (ICM) capable of sensing and recording electrocardiogram (ECG) signals from a position outside of heart 104 via electrodes (not shown in FIG. 1). In some examples, IMD 101C includes or is coupled to one or more additional sensors, such as accelerometers, that generate one or more signals that vary based on patient motion and/or posture, blood flow, or respiration. Examples of IMD 101C may monitor a physiological parameter indicative of patient state, such as posture, heart rate, activity level, and/or respiration rate. IMD 101C may be implanted outside of the thorax of patient 102, e.g., subcutaneously or sub-muscularly, such as the pectoral location illustrated in FIG. 1. In some examples, IMD 101C may take the form of a Reveal LINQ® ICM, available from Medtronic plc, of Dublin, Ireland.

In various examples, an IMD 101 is configured to wirelessly communicate with one or more external device(s) 110 as illustratively shown in FIG. 1 by communication link 112. External device(s) 110 may be a computing device, e.g., used in a home, ambulatory, clinic, or hospital setting, to wirelessly communicate with an IMD 101. For example, external device(s) 110 may be a patient monitor, such as the MyCareLink™ patient monitor, or a programming instrument, such as the Care Link SmartSync™ system, available from Medtronic Inc., a subsidiary of Medtronic plc of Dublin, Ireland. In another example, external device(s) 110 may be a mobile computing device such as a smartphone, tablet, smartwatch, or other wearable or portable device. External device(s) 110 may, for example, include a mobile application, such as MyCareLink Heart™ mobile app, available from Medtronic Inc., a subsidiary of Medtronic plc of Dublin, Ireland, that enables external device(s) 110 to communicate with IMD 101. External device(s) 110 may be coupled to a remote patient monitoring system, such as CareLink™ Network, available from Medtronic Inc., a subsidiary of Medtronic plc, of Dublin, Ireland. External device(s) 110 may be, as examples, a programmer, external monitor, or consumer device, e.g., smart phone. For example, external device(s) 110 may be used to program commands or operating parameters into IMDs 101 for controlling the functioning of that IMD. External device(s) 110 may be used to interrogate an IMD 101 to retrieve data, including device operational data as well as physiological or neurological data accumulated in memory of the IMD. The interrogation may be automatic, e.g., according to a schedule, or in response to a remote or local user command. One or more of these external device(s) 110 may also be referred to as an “instrument” or as a group of instruments.

Examples of communication techniques used by IMDs 101 and external device(s) 110 include Bluetooth Low Energy communication protocols, but are not limited to any particular communication technique or communication protocol. In other examples, tissue conductance communication (TCC) or RF telemetry, which may be an RF link established via Bluetooth®, WiFi, or medical implant communication service (MICS) may be used. The IMDs 101 may utilize an antenna arranged as described in this disclosure, or an equivalent thereof, to perform the communications associated with the IMD, in order to provide any of the features and to perform any of the functions ascribed to that IMD.

In various examples, one or more of the IMDs 101 in FIG. 1 may include the antenna arranged in accordance with the examples of antenna described in this disclosure, and any equivalents thereof, to facilitate communications with the one or more IMDs 101 of system 100, and/or between the one or more IMDs 101, and/or external device(s) 110.

For the remainder of the disclosure, a general reference to a medical device system may refer collectively to include any examples of medical device system 100, as described above with respect to FIG. 1, and any equivalents thereof. Further, for the remainder of the disclosure a general reference to an IMD may refer collectively to include any examples of IMD 101A, IMD 101B, and/or IMD 101C, as described above with respect to FIG. 1, and any equivalents thereof.

The example IMDs of FIG. 1 include a housing configured to house a communication circuit and at least one of stimulation and sensing circuitry within an internal side of the housing. For example, a battery such as lithium/iodine cell may be coupled to a motherboard or flex circuit that hosts one or more semiconductor chips and other electronic circuitry. In some examples, the communication circuit and/or stimulation and sensing circuitry may be part of the one or more semiconductor chips.

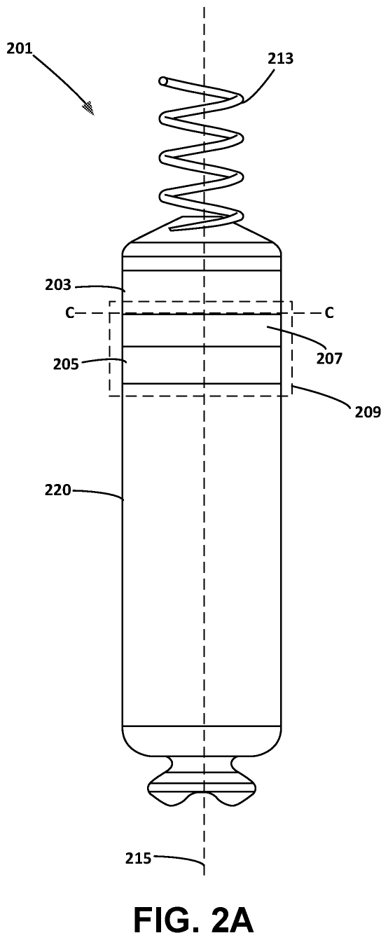

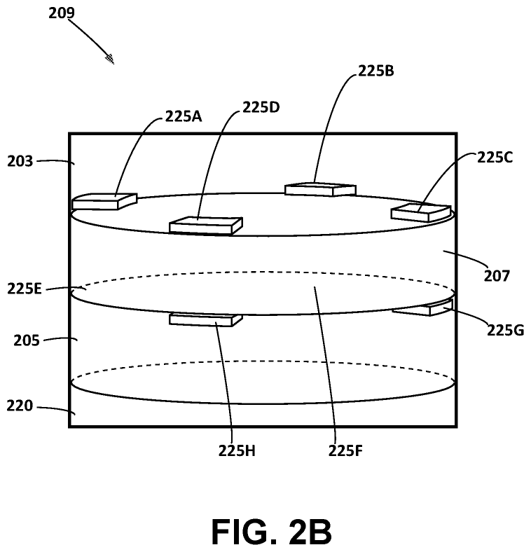

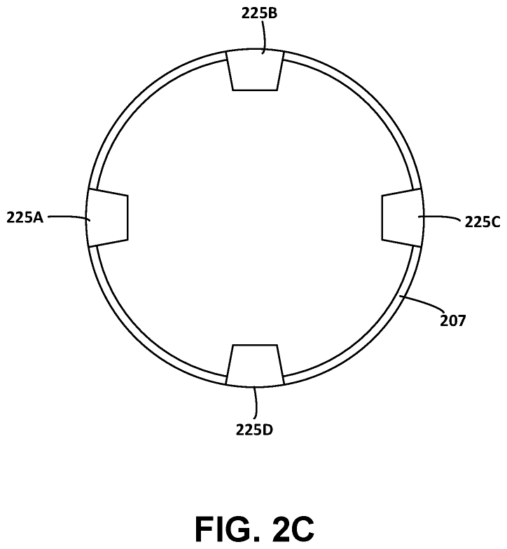

FIG. 2A illustrates an example of IMD 201 in accordance with techniques of this disclosure. FIG. 2B is a diagram illustrating a portion of IMD 201, corresponding to box 209 in FIG. 2A, in greater detail. FIG. 2C is a cross-sectional diagram of IMD 201 taken along line C-C in FIG. 2A, viewed in the direction of housing portion 220, and with certain components within the interior space defined by metal portions 203 and 205 and non-conductive component 207 removed.

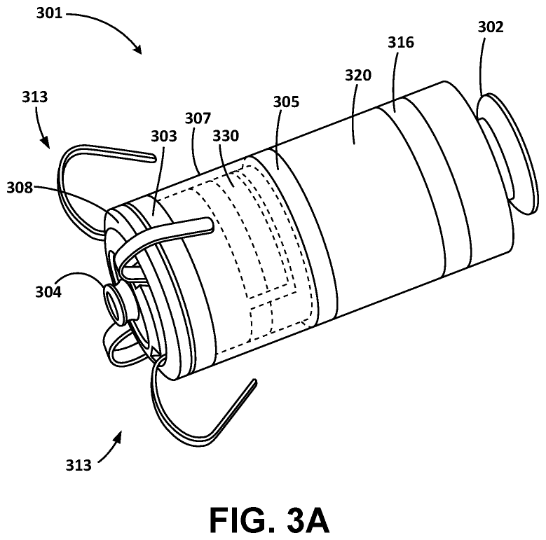

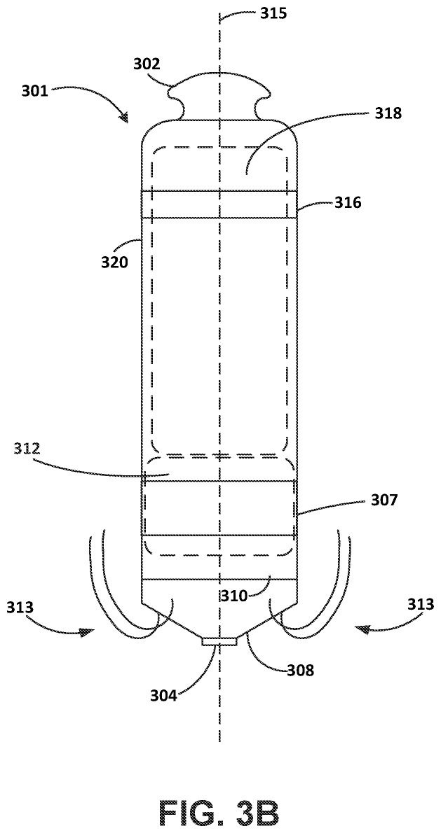

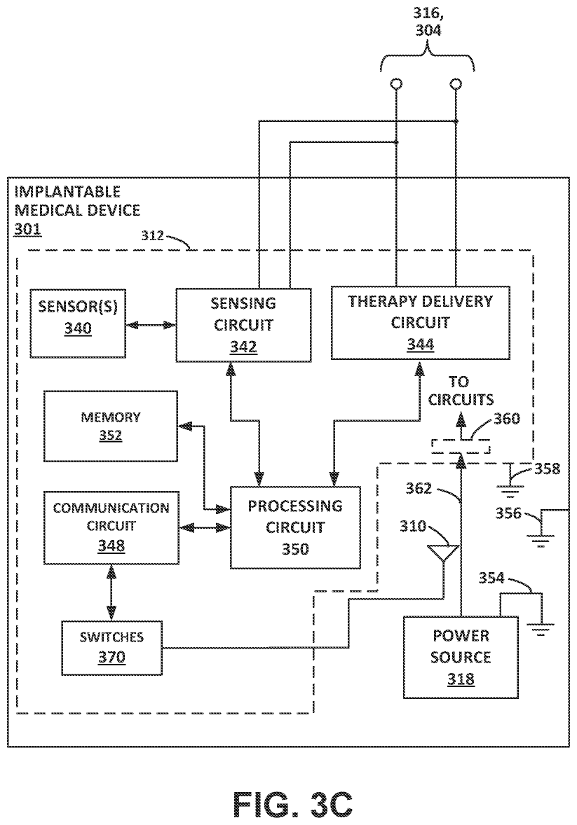

IMD 201 may be an example of IMD 101A of FIG. 1, e.g., an intracardiac pacing device, such as a Medtronic Micra™ Transcatheter Pacing System. As shown, IMD 201 may include generally cylindrical housing including housing portion 220, ring-shaped non-conductive component 207, and ring-shaped metal portions 203 and 205. Housing portion 220 may house a battery of IMD 201, while electronics of IMD 201, such as a communication circuit, are housed inside the interior space bounded by non-conductive component 207 and ring-shaped metal portions 203 and 205, and an upper surface of housing portion 220. IMD 201 may include fixation mechanism 213, which may be helical as shown in FIG. 2A and acts to hold the IMD in place by attaching it to heart tissue. Fixation mechanism 213, or a portion thereof, may act as an electrode for sensing cardiac signals and delivering pacing pulses. In some examples, fixation mechanism 213 includes one or more darts, or one or more tines as shown in FIGS. 3A and 3B. In some examples, fixation mechanism 213 includes multiple components of one or more types, such as multiple helices, multiple tines, or multiple darts.

Housing portion 220 and ring-shaped metal portions 203 and 205 may be, but are not necessarily, formed of a common metal, such as titanium, cobalt, chromium, nickel, alloys thereof, or stainless steel. Non-conductive component 207 and portions 203, 205, 220 have a common axis, which is the longitudinal axis 215 of IMD 201. Portions 203 and 205 may function as ground planes and feed points on each side of non-conductive component 207. Non-conductive component 207 may be formed of a material that is transparent, or at least partially transparent, to RF waves, such as sapphire or ceramics, e.g., alumina ceramics or zirconia ceramics. Ring-shaped non-conductive components, such as non-conductive component 207 may comprise a continuous ring, tube or cylinder of non-conductive material, or may comprise a plurality of segments of non-conductive material arranged in a ring, e.g., around longitudinal axis 215. Segments of non-conductive component 207 may be separated, e.g., by the same material from which metal portions 203 and 205 are formed, or other material(s). In some examples, rather than a full ring, a non-conductive component of an antenna according to this disclosure may be formed as a partial ring. In some examples, a non-conductive component of an antenna according to this disclosure may be formed as a “window” in a housing of an IMD, having an outer boundary shape that may be polygonal or elliptical, as examples.

IMD 201 may also include a plurality of contacts or feed points 225A-225H (collectively, “contacts 225”), which may act as signal feed lines for coupling the antenna 211, via metal portions 203 and 205, to a communication circuit within the housing. In some examples, two or more of contacts 225 are located proximate to each of the sides or ends of non-conductive component 207. In some examples, contacts 225 on opposite sides of non-conductive component 207 are arranged as pairs, e.g., each on a respective side. In some examples, contacts 225 are distributed, e.g., evenly, about a lateral periphery, e.g., circumference, of IMD 201. For example, with reference to FIG. 2B, the size and shape of contacts 225 in FIGS. 2B and 2C are for purposes of illustration, and contacts 225 may have any size/shape configured to provide an electrical connection.

The non-conductive component 207 and metal portions 203 and 205, together with the contacts 225, form antenna 211, which is configured to be excited to provide RF wireless communications, in conjunction with the communication circuit, between IMD 201 and one or more other devices. IMD 201, e.g., the communication circuit, may include switches respectively associated with one or more contacts 225. For example, based on the configuration of the switches, each contact 225 may be toggled between short, open, or connected to an RF signal feed, as described more fully below. In some examples, a subset of contacts 225 may not be switchable, and may be fixed in one of the short, open, or connected states, e.g., one or more fixed shorts and one or more fixed feed points. In some examples, a subset of contacts 225 are fixed as shorts, while other contacts are switchable between open or connected to configure an excitation pattern. Where arranged as pairs on opposite sides of non-conductive component 207, the contacts 225 of a pair may be commonly switched/configured, e.g., the pair of 225D and 225H may both be connected, both be open, or both be short. When a pair of contacts 255 are commonly switched/configured to be connected (e.g., as signal input and ground), they may act to drive metal portions 203 and 205 to excite antenna 211 with non-conductive component 207 acting as a gap between metal portions 203 and 205. In some examples, to excite antenna 211, contacts 225 are configured to provide an input and ground on opposite sides of non-conductive component 207, and one or more switched or fixed shorts across non-conductive component 206 define a path for an RF current signal to flow around the non-conductive component.

Based on selection of different configurations of the switches, multiple modes of excitation may be provided in order to provide spatial diversity for the RF signal. In one example, pairs 225D/225H and 225B/225F, 180 degrees from each other, may be configured as short, and pairs 225A/225E and 225C/225G, 180 degrees from each other, may be connected to the signal. One of pairs 225A/225E or 225C/225G may be selected to provide directionality of the RF signal. The wireless communications may be Bluetooth Low Energy (BLE) signals, or another communication protocol as previously described.

FIG. 3A illustrates an example IMD 301 in accordance with techniques of this disclosure. IMD 301 may also be an example of IMD 101A of FIG. 1. Like numbered elements of IMD 301 may be configured substantially similarly to their counterparts of IMD 201 in FIGS, 2A-2C, except as noted herein.

IMD 301 may include a housing including a generally cylindrical portion 320, a ring-shaped (e.g., tubular or cylindrical) non-conductive component 307, and ring-shaped (e.g., tubular or cylindrical) metal portions 303 and 305 which have a common axis. The sizes of non-conductive components 207, 307 illustrated in FIGS. 2A-3A are merely examples. In the longitudinal direction, a length of non-conductive components 207, 307 may be within a range from 1 millimeter (mm) to 5 mm, such as about 2 mm. A diameter of non-conductive components 207, 307 may be within a range from 3 mm to 9 mm, such as within a range from 5 mm to 7 mm. A communication range of antenna 211, 311 may be within a range from 1 meter to 10 meters.

Portions 303 and 305 may function as ground planes and feed points on each side of non-conductive component 307. The antenna including non-conductive component 307 may include contacts not shown in FIG. 3A, e.g., contacts 225 of FIGS. 2B and 2C. The housing also includes a header 308 or cap portion. A fixation mechanism 313, e.g., a plurality of tines, extend from header 308, and is configured to hold IMD 301 in place within a patient's heart. Flange 302 is configured to allow one or more instruments to be attached to IMD 301 during implantation/retrieval of IMD 301.

IMD 301 includes electrodes 304 and 316, which may be formed integral with the housing. In the example of FIG. 3A, electrode 304 is disposed on a distally extending projection of header 308, and electrode 316 is a portion, e.g., an insulated portion, of cylindrical portion 320. IMD 301 senses electrical activity of the patient's heart, and delivers pacing pulses to the patient's heart, via electrodes 304 and 316.

In the example of FIG. 3A, IMD 301 includes a flex circuit 330. As illustrated in FIG. 3A, flex circuit 330 may be disposed within housing, e.g., beneath non-conductive component 307, such that it substantially conforms to the inner surface of non-conductive component 307, e.g., is wrapped/bent into a ring shape. In some examples, flex circuit 330 may be maintained in contact, e.g., electrical contact, with non-conductive component 307 and or metal portions 303 and 305. For example, flex circuit 330 may be glued or bonded to one or more of these elements. In some examples, a mechanism, e.g., compressible material on the inner surface of flex circuit 330, may bias flex circuit 330 radially outward against these components. In some examples, flex circuit 330 may include a mechanism, e.g., a spring or other elastically compressible element on the outer surface of flex circuit 330, which may flexibly maintain electrical contact between circuit 330 and metal portions 303 and 305.

Flex circuit 330 may include tracings that function as contacts 225 (FIGS. 2B and 2C), and which together with dielectric resonator 307 function as an antenna to provide RF wireless communications. In some examples, flex circuit 330 includes or is coupled to the switches that selectively couple the contacts to the communication circuit of IMD 301. Switching which contacts, e.g., which portion of flex circuit 330, is coupled as a feed for the communication signal to the antenna, may provide directionality of the emitted signal. In some examples, flex circuity 330 includes some of all of the communication circuit. Flex circuit 330 may include an RF system on a chip. In some examples, flex circuit 330 includes additional circuitry of IMD 301, e.g., as described with respect to FIG. 3C.

FIG. 3B is a conceptual diagram further illustrating example components of IMD 301, according to techniques described in this disclosure. IMD 301 includes dielectric resonator 307 (ground planes and contacts omitted for clarity in FIG. 3B) of a DRA 310. IMD 301 also includes power source 318, e.g., within housing portion 320, and electronic circuitry 312, e.g., within the portion of the housing that is defined in part by non-conductive component 307. Power source 318 may be a battery that is coupled to and provides power to the electronic circuitry 312. IMD 301 defines longitudinal axis 315.

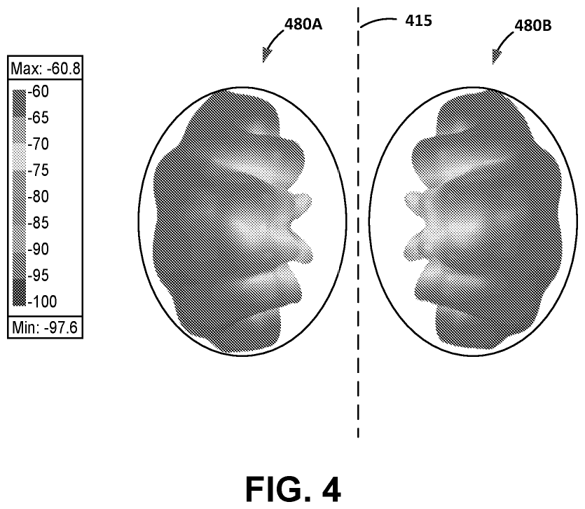

Electronic circuitry 312 may include components on flex circuit 330 and/or components, e.g., on one or more boards or substrates, within the space in housing defined by header 308, housing portions 303 and 305, non-conductive component 307, and housing portion 320. Traces on the flex circuit may provide signal lines to the contacts 225, and thus antenna 310. A flex circuit may also include other circuit components such as an RF SOC (system on a chip), RF switches, components such as described above, and the like. A communication circuit (such as communication circuit 348 shown in FIG. 3C) may be configured to provide excitation of antenna 310 and communication signals for wireless communications between IMD 301 and one or more external devices and/or other IMDs as described with respect to FIG. 1. Antenna 310 is configured to provide efficient wireless communication using a predetermined frequency or range of frequencies, such as BLE communication frequencies. Performance wise, antenna 310 may have radiation efficiency and gain that is comparable to much larger devices at a similar implant depth, e.g., as illustrated with respect to FIGS. 4-6.

As one example, contacts 225 may be configured to act as RF switches by being at times coupled to communication circuit 348 and at times coupled to ground (e.g., metal of housing) or opened and then toggled between the two states. In this manner, the antenna 310 may be excited and RF signals routed through the antenna. Different excitation modes may be selected using different switching patterns and switching frequencies.

Communication circuit 348 may include a transmitter, a receiver, or a transceiver. That is, communication circuit 348 may provide for bi-directional communication (e.g., transmit and receive communication) or uni-directional communication (e.g., receive but not transmit data or transmit by not receive data). Communication circuit 348 may be configured to output a modulated signal that causes antenna 310 to radiate an electromagnetic signal carrying the data that IMD 301 is to transmit. For receiving, an electromagnetic signal may cause a signal antenna 310 that communication circuit 348 receives and demodulates to determine the data that is transmitted to IMD 301.

Antenna 310 may be configured to communicate in accordance one or more wireless communication protocols such as Bluetooth®, WiFi, or medical implant communication service (MICS). That is, antenna 310 may be configured to have a resonant frequency that is approximately equal to the frequency (or frequencies) used for one or more example communication protocols. Here, approximately refers to the resonant frequency of antenna 310 being within the range of frequencies that conform to the example communication protocols.

FIG. 3C is a functional block diagram illustrating an example configuration of IMD 301, according to techniques described in this disclosure. IMD 201 of FIGS. 2A-2C may be similarly configured.

As shown in FIG. 3C, IMD 301 includes a power source 318 that is coupled to the electronic circuitry 312 provided in IMD 301, and is configured to provide electrical power to the components of electronic circuitry 312. Electronic circuitry 312 as shown in FIG. 3C includes processing circuit 350, memory 352, sensing circuit 342, therapy delivery circuit 344, and sensor(s) 340. Sensor(s) 340 may include one or more position and/or motion sensing sensors, such as accelerometers, and/or other physiological sensors. Electronic circuitry 312 also includes communication circuit 348 coupled to antenna 310.

The electronic circuitry 312 and devices included in electronic circuitry 312 may be provided on one or more structures, such as a flex circuit 330, one or more printed circuit boards, or the like. As shown in FIG. 3C, power source 318 includes a power connection 362 that is electrically connected to one terminal (voltage level) of power source 318. Electrical contact 360 may be electrically coupled to the electrical conductors and electronic components of electronic circuitry 330 and configured to provide a flow of current provided by power source 318 and flowing through power connection 362 to electronic circuitry 330. In various examples, connection 358 may be electrically coupled to a reference voltage (second terminal) of power source 318, to provide one return path for current provided to electronic circuitry 330 from power source 318. In various examples, connection 358 is coupled to the housing 320 of IMD 301, wherein the housing is coupled to reference voltage 354 of power source 318, thus using the housing of IMD 301 as a return path for current provided to power electronic circuitry 330 from power source 318.

In the illustrated example, IMD 301 includes processing circuit 350 and an associated memory 352, sensing circuit 342, therapy delivery circuit 344, one or more sensors 340, and the communication circuit 348 coupled to antenna 310 via switches 370, as described above. However, IMD 301 need not include all of these components, or may include additional components. For example, some examples of IMD 301 that do not provide therapy, such that therapy delivery circuit 344 may not be included in IMD 301.

Memory 352 includes computer-readable instructions that, when executed by processing circuit 350, cause IMD 301 and processing circuit 350 to perform various functions attributed to IMD 301 and processing circuit 350 herein, (e.g., preparing and transmitting from IMD 301 information and data by wireless communication using communication circuit 348 and antenna 310 prepared by processing circuit 350, and receiving at antenna 310 and through communication circuit 348, wireless communications, and processing the received communications for example using processing circuit 350). Memory 352 may include any volatile, non-volatile, magnetic, optical, or electrical media, such as a random-access memory (RAM), read-only memory (ROM), non-volatile RAM (NVRAM), electrically-erasable programmable ROM (EEPROM), flash memory, or any other digital or analog media. Memory 352 may store threshold(s) for time of day, posture, heart rate, activity level, respiration rate, and other parameters. Memory 352 may also store data indicating cardiovascular pressure measurements. Memory 352 may store data, instructions, and/or parameters for use by processing circuit 350 and/or communication circuit 348 in performing the telemetry and communication functions of the IMD. Processing circuit 350 may be configured to access data and/or instructions stored in memory 352 in order to perform any of the function and provide any of the features ascribed to IMD 301 throughout this disclosure, and any equivalents thereof.

Processing circuit 350 may include fixed function circuitry and/or programmable processing circuitry. Processing circuit 350 may include any one or more of a microprocessor, a controller, a digital signal processor (DSP), an application specific integrated circuit (ASIC), a field-programmable gate array (FPGA), or equivalent discrete or analog logic circuitry. In some examples, processing circuit 350 may include multiple components, such as any combination of one or more microprocessors, one or more controllers, one or more DSPs, one or more ASICs, or one or more FPGAs, as well as other discrete or integrated logic circuitry. The functions attributed to processing circuit 350 herein may be embodied as software, firmware, hardware or any combination thereof.

Sensing circuit 342 and therapy delivery circuit 344 are coupled to electrodes 304 and 316 of IMD 301. Sensing circuit 342 may monitor signals from electrodes 304, 316 in order to monitor electrical activity of heart, impedance, or some other electrical phenomenon. Sensing of a cardiac electrical signal may be done to determine heart rates or heart rate variability, or to detect arrhythmias (e.g., tachyarrhythmias or bradycardia) or other electrical signals. In some examples, sensing circuit 342 may include one or more filters and amplifiers for filtering and amplifying a signal received from electrodes 304, 316. In some examples, sensing circuit 342 may sense or detect physiological parameters, such as heart rate, blood pressure, respiration, and other physiological parameters associated with a patient.

The resulting cardiac electrical signal may be passed to cardiac event detection circuitry that detects a cardiac event when the cardiac electrical signal crosses a sensing threshold. The cardiac event detection circuitry may include a rectifier, filter and/or amplifier, a sense amplifier, comparator, and/or analog-to-digital converter. Sensing circuit 342 outputs an indication to processing circuit 350 in response to sensing of a cardiac event (e.g., detected P-waves or R-waves).

In the example of FIG. 3C, IMD 301 includes one or more sensors 340 coupled to sensing circuit 342. Although illustrated in FIG. 3C as included within IMD 301, one or more of sensors 340 may be external to IMD 301, e.g., coupled to IMD 301 via one or more leads, or configured to wirelessly communicate with IMD 301. In some examples, sensors 340 transduce a signal indicative of a patient parameter, which may be amplified, filtered, or otherwise processed by sensing circuit 342. In such examples, processing circuit 350 determines values of patient parameters based on the signals. In some examples, sensors 340 determine the patient parameter values, and communicate them, e.g., via a wired or wireless connection, to processing circuit 350.

Therapy delivery circuit 344 is configured to generate and deliver electrical therapy to the heart. Therapy delivery circuit 344 may include one or more pulse generators, capacitors, and/or other components capable of generating and/or storing energy to deliver as pacing therapy, defibrillation therapy, cardioversion therapy, other therapy or a combination of therapies. In some instances, therapy delivery circuit 344 may include a first set of components configured to provide pacing therapy and a second set of components configured to provide anti-tachyarrhythmia shock therapy. In other instances, therapy delivery circuit 344 may utilize the same set of components to provide both pacing and anti-tachyarrhythmia shock therapy. In still other instances, therapy delivery circuit 344 may share some of the pacing and shock therapy components while using other components solely for pacing or shock delivery.

Therapy delivery circuit 344 may include charging circuitry, one or more charge storage devices, such as one or more capacitors, and switching circuitry that controls when the capacitor(s) are discharged to electrodes. Charging of capacitors to a programmed pulse amplitude and discharging of the capacitors for a programmed pulse width may be performed by therapy delivery circuit 344 according to control signals received from processing circuit 350, which are provided by processing circuit 350 according to parameters stored in memory 352. Processing circuit 350 controls therapy delivery circuit 344 to deliver the generated therapy to the heart via one or more combinations of electrodes, e.g., according to parameters stored in memory 352. Therapy delivery circuit 344 may include switch circuitry to select which of the available electrodes are used to deliver the therapy, e.g., as controlled by processing circuit 350.

Communication circuit 348 includes any suitable hardware, firmware, software or any combination thereof for communicating with another device, such as an external device, or another IMD or sensors, such as IMDs shown in FIG. 1. Under the control of processing circuit 350 as shown in FIG. 3C, communication circuit 348 may receive downlink telemetry from and send uplink telemetry to external device or another device with the aid of antenna 310 which may be arranged according to any of the example antennas described herein, or any equivalents thereof. In some examples, communication circuit 348 may communicate with a local external device, for example through one or more of external device 110 (FIG. 1), and processing circuit 350 may communicate with a networked computing device via the local external device and a computer network, such as the Medtronic® CareLink® Network developed by Medtronic, ple, of Dublin, Ireland.

A clinician or other user may retrieve data from IMD 301 using external device(s) 110 (FIG. 1), or another local or networked computing device configured to communicate with processing circuit 350 via communication circuit 348. The clinician may also program parameters of IMD 301 using external device(s) 110 (FIG. 1) or another local or networked computing devices.

In various examples, processing circuit 350 is configured to receive signals from sensing circuit 342, sensors 340, and or sensor signal provided by sensors external to IMD 301, to process these sensor signals to generate one or more input parameters based either directly on or derived from the sensor signals. The input parameters are associated with current value(s) for one or more physiological parameters associated with a patient. The physiological parameters associated with the input parameters may include activity counts, respiration rates, breathing rates, movements, postures, and changes in postures associated with a patient. The current values associated with these input parameters can be values measured directly from the input parameters, or derived for these input parameters. For example, a value of a heartrate, measured for example in heartbeats per minute or cardiac cycle length, may be determined as the current value (e.g., the most recent value) for the input parameter associated with the heart rate of the patient measured over some predefined time period. Similarly, a value of a breathing rate, measured for example in breaths per minute or breathing cycle length, may be determined as the current value (e.g., the most recent value) for the input parameter associated with the breathing rate of the patient as measured over some predefined time period.

Similarly, current values can be determined for other input parameters, such as activity count (e.g., based on movement of the patient measured for example in steps taken by the patient per minute), body temperature, and for example a current value for a posture of the patient (e.g., lying down, standing, sitting). A current value of a physiological parameter may be, in some examples, a mean or median of measured values over a period of time. These sensed and determined parameters associated with the patient may be used to control the therapy delivery circuit 344 in providing electrical stimulation therapy, for example pacing and/or shock therapy, to the patient.

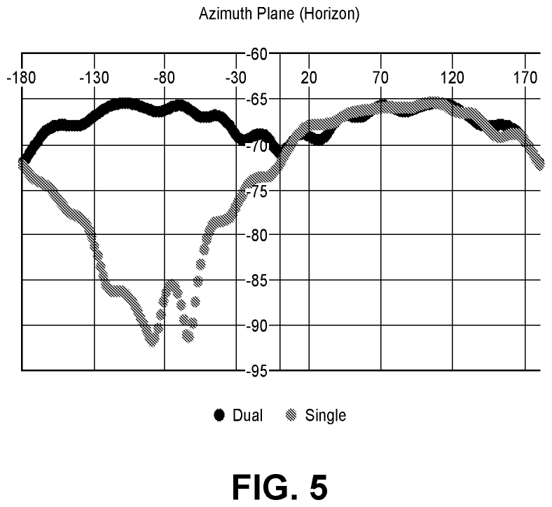

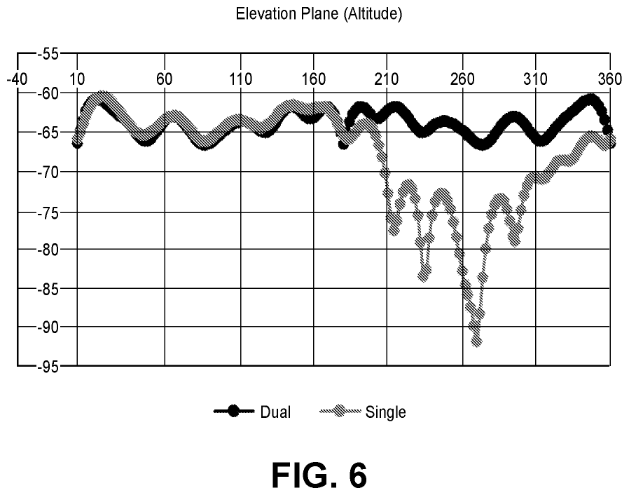

FIG. 4 is a conceptual diagram illustrating an example of radiation performance for antenna 310 for two different modes of antenna excitation, such as by using different switching patterns for the switches, wherein the two different modes produce radiation patterns that are spatially complimentary to each other. This dual mode operation of excitation illustrates a spatial diversity in radiation pattern relative to a longitudinal axis 415 of an IMD, which may be similar to longitudinal axes 215 and 315 shown in FIGS. 2A and 3B. In particular, FIG. 4 illustrates an example in which feeding signal to antenna 310 via each of two locations that are on opposite sides of a longitudinal plane including longitudinal axis 415 allows the resulting radiation to be beamformed to a respective half of a transverse plane through the longitudinal plane. In some examples, antenna 310 may comprise two sub-antennas with respective feed locations. By operating the antenna in a dual mode with complimentary modes of excitation, an RF radiation signal can be approximately constant in all directions, such as illustrated in more detail in FIG. 5 and FIG. 6. In some examples, a processing circuit of an IMD, or of external device in communication with the IMD, may conserve energy of the IMD by selecting one feed location or the other based on which part (e.g., half) of antenna 310 provides better (or acceptable) communication performance (e.g., based on an orientation of the IMD relative to the patient or the external computing device).

Specifically, these figures show spatial gain comparisons for an antenna that is excited in a dual mode vs. an antenna that is excited in a single mode, in an azimuth plane and an elevation plane. The single mode results illustrated in FIG. 5 and FIG. 6 reflect the effective absence of one of directions or lobes 480A and 480B from the radiation pattern shown in FIG. 4 due to excitation via switches of feed location(s) on one circumferential half of the IMD, e.g., excitation of one sub-antenna. By using dual mode excitation (i.e., with spatial diversity), radiation nulls may be essentially eliminated, such that radiation may be emitted in an approximately uniform manner in all directions. In this manner, a radiation gain of an RF wireless communication to an external device is roughly the same regardless of an orientation of the IMD when implanted in a patient. In some examples, however, communication quality feedback may be used to select a single excitation mode, e.g., corresponding to one of lobes 480A or 480B by selecting one feed location or another via switches as described above, that provides adequate quality. In this manner, increased power consumption associated with driving the entire antenna to provide dual mode operation can be avoided.

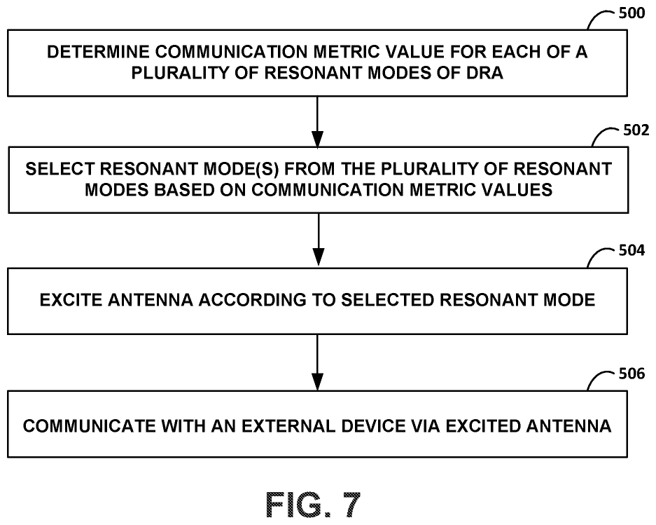

FIG. 7 is a flow diagram illustrating an example operation of an IMD, e.g., IMD 301, including an antenna, e.g., antenna 310, in accordance with one or more techniques described in this disclosure. According to the example of FIG. 7, processing circuit 350 of IMD 301 may determine a value of a communication metric for two or more of a plurality of excitation modes of antenna 310, e.g., for two spatial excitation modes corresponding to the example of FIGS. 4-6 (500). In some examples, processing circuit 350 may test excitation modes by controlling communication circuit 348 to excite antenna 310 with a plurality of configurations of switches 370 and determining corresponding metric values, e.g., ability to make a connection, or signal strength, packet rate, packet error rate, bit rate, bit error rate, or another metric of link quality if the connection is made. In some examples, the excitation modes may include the two modes that respectively feed different physical portions (e.g., halves or sides relative to a longitudinal axis) of the antenna to respectively produce a radiation pattern including one of lobes 480A and 480B in FIG. 4 and, in some cases, the dual mode feeding both locations to produce a radiation pattern corresponding to both of lobes 480A and 480B. In some examples, processing circuitry 350 may control each single excitation mode of antenna 310 by controlling communication circuitry 348 to drive a pair of contacts 225 on opposite sides of a non-conductive component 307 (as signal and ground), with circumferentially adjacent contacts shorted across the non-conductive component, e.g., shorts across the non-conductive component spaced about 90 degrees from the driven contact pair. Processing circuitry 350 may determine the communication metric values via communication with an external device 12.

Processing circuit 350 selects one of the excitation modes based on the metric values, e.g., based on the corresponding metric value satisfying a threshold or being the “best” (highest or lowest) of the determined values (502). Processing circuit 350 controls communication circuit 348 to excite antenna 310 according to the selected excitation mode, e.g., via an associated configuration of switched 370(504 ). IMD 301 communicates with an external device 12 via the excited antenna (506).

The example operation of FIG. 7 may be performed at various times. In some examples, the operation of FIG. 7 may be performed during an implantation procedure for IMD 10, e.g., to identify an excitation mode to be subsequently used by IMD 10. In some examples, the orientation of IMD 10 may be relatively stable after implantation.

In some examples, multiple excitation modes may be identified for use in certain conditions, and processing circuit 350 of IMD 10 may automatically switch between such modes based on detection of such conditions, e.g., times of day, postures or activity levels of the patient, e.g., as indicated by an accelerometer or other sensor 340, or phases of the cardiac cycle of the patient. In some examples, processing circuit 350 may perform the operation of FIG. 7 or otherwise automatically switch to a different excitation mode based on detection of loss of communication or inability to establish communication.

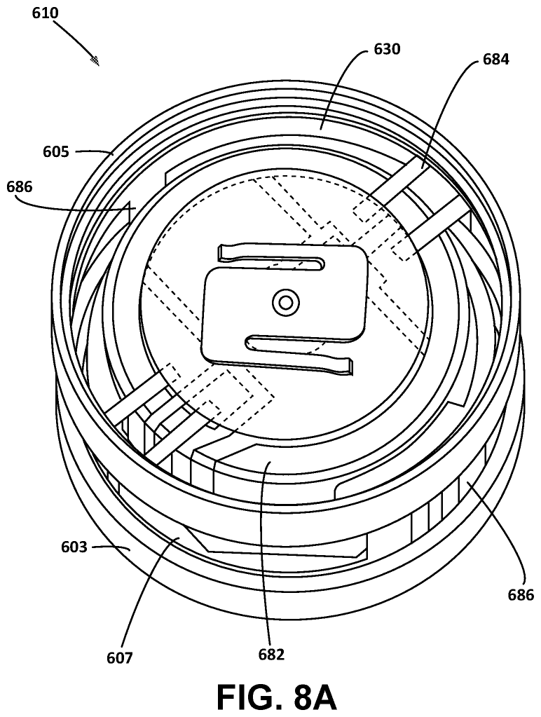

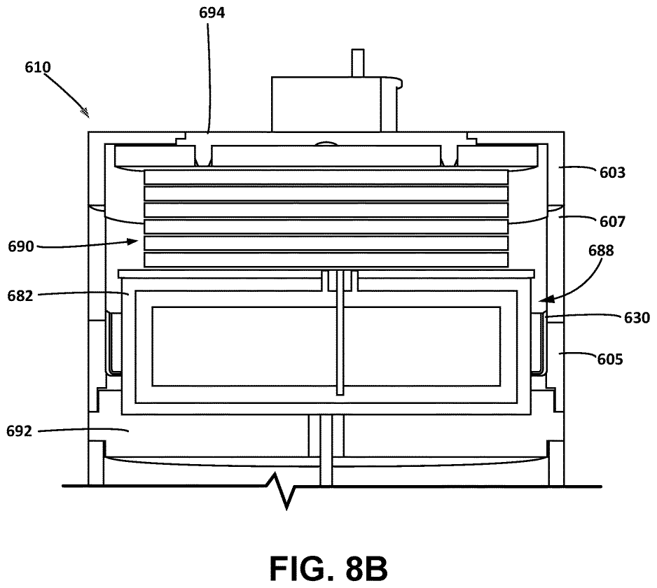

FIGS. 8A and 8B are perspective and cross-sectional diagrams illustrating an example antenna 610 and an interconnect device 682 of an IMD in accordance with techniques described in this disclosure. As illustrated in FIGS. 8A and 8B, antenna 610 may include a non-conductive component 607 between adjacent metal portions 603 and 605 on opposite longitudinal sides of non-conductive component 607, similar to metal portions 203,303 and 205,305 and non-conductive components 207,307 of FIGS. 2A-2C and FIGS. 3A and 3B. Antenna 610 also includes one or more flex circuits 630 configured to provide electrical contacts to metal portions 603 and 605.

In the example illustrated by FIGS. 8A and 8B, the IMD includes an interconnect device 682 and circuitry 690, e.g., on one or more stacked printed circuit boards, within the interior space within the housing of the IMD defined by metal portions 603 and 605 and non-conductive component 607. Interconnect device 682 may provide electrical paths, e.g., traces, to connect metal portions 603 and 605 to a communication signal from communication circuit 348 and electrical ground. Interconnect device 682 may be a molded device configured to include such metallic connection components. In some examples, one or more circuit boards of circuitry 690 may be unstacked within the interior space within the housing of the IMD defined by metal portions 603 and 605 and non-conductive component 607.

There may be a gap 688 between interconnect device 682 and flex circuit 630 or between flex circuit 630 and metal portions 603 and 605, which may be bridged by elastically deformable connectors 684, e.g., conductive springs. In some examples, instead of or in addition to flex circuit 630 and/or connectors 684, metal portions 603 and 605 may be connected to circuitry 690 and ground via other portions of the housing of the IMD electrically common with metal portions 603 and 605, such as portion 694 and portion 692, respectively. FIG. 8A also illustrates electrical shorts 686 that may electrically isolate portions of antenna 610, e.g., so that each portion may separately act as a respective directional antenna when excited individually. In some examples, the two shorts 686 illustrated in FIG. 8A electrically isolate hemispheres of antenna 610 to facilitate the two single excitation modes and the dual excitation mode described with respect to FIGS. 4-6.

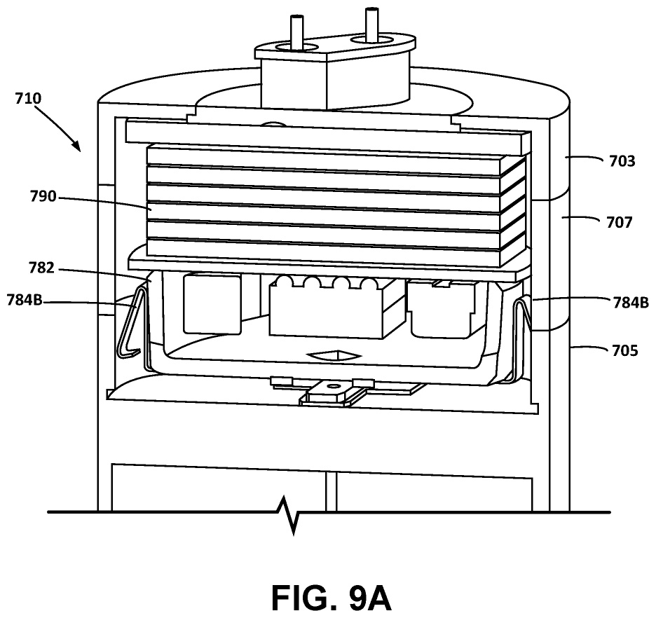

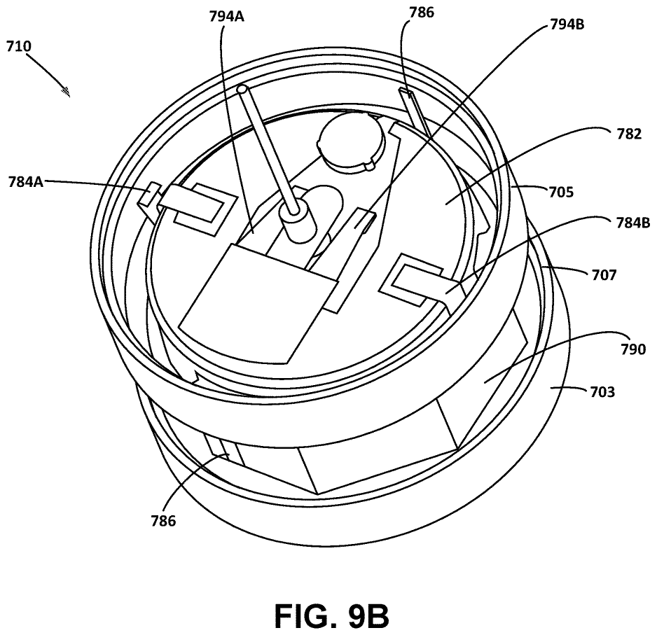

FIGS. 9A and 9B are cross-sectional and perspective diagrams, respectively, illustrating another example antenna 710 and another example interconnect device 782 of an IMD in accordance with techniques described in this disclosure. As illustrated in FIGS. 9A and 9B, antenna 710 may include a non-conductive component 707 between adjacent metal portions 703 and 705 on opposite longitudinal sides of non-conductive component 707, similar to metal portions 203,303 and 205,305 and non-conductive components 207,307 of FIGS. 2A-2C and FIGS. 3A and 3B. The IMD also includes an interconnect device 782 and circuitry 790, similar to interconnect device 682 and circuitry 690 described above with respect to FIGS. 8A and 8B. Antenna 710 does not necessarily include one or more flex circuits configured to provide electrical contacts to metal portions 703 and 705. In some examples, one or more circuit boards of circuitry 790 may be stacked within the interior space within the housing of the IMD defined by metal portions 703 and 705 and non-conductive component 707. In some examples, one or more circuit boards of circuitry 790 may be unstacked within the interior space within the housing of the IMD defined by metal portions 703 and 705 and non-conductive component 707.

FIG. 9B illustrates electrical shorts 786 that may electrically isolate portions of antenna 710, e.g., so that each portion may separately act as a respective directional antenna when excited individually. Shorts 786 may provide the functionality with respect to antenna 710 as described above with respect to shorts 686 of antenna 610 and FIG. 8A. FIG. 9B also illustrates elastically-deformable connectors 794A and 794B (collectively, “connectors 794”) for redundantly connecting a battery or other power source of the IMD to circuitry 790 via interconnect device 782.

Additionally, as illustrated in FIGS. 9A and 9B, the IMD may include elastically deformable connectors 784A and 784B (collectively, “connectors 784”). Connectors 784 may be fixedly connected to interconnect device 782 and configured to make electrical and physical contact with metal portion 705. Connectors 784 are configured, e.g., due to their material and the manner in which they are bent or otherwise shaped, to be spring-like and elastically deform, e.g., inward toward a longitudinal axis of the IMD when interconnect device 782 is inserted into housing and connectors 784 engage metal portion 705. Connectors 784 may be considered e.g., conductive springs.

Each of connectors 784 may be selectively coupled to a respective section, e.g., circumferential section, of metal portion 705, electrically separated from other sections of metal portion 705 by two shorts 686. In the illustrated example, two shorts 686 separate antenna 710, including metal portion 705, into two hemispherical sections or sub-antenna. Each of connectors 784A and 784B may be respectively connected the RF signal, e.g., via interconnect device 782, to excite its respective section of antenna 710.

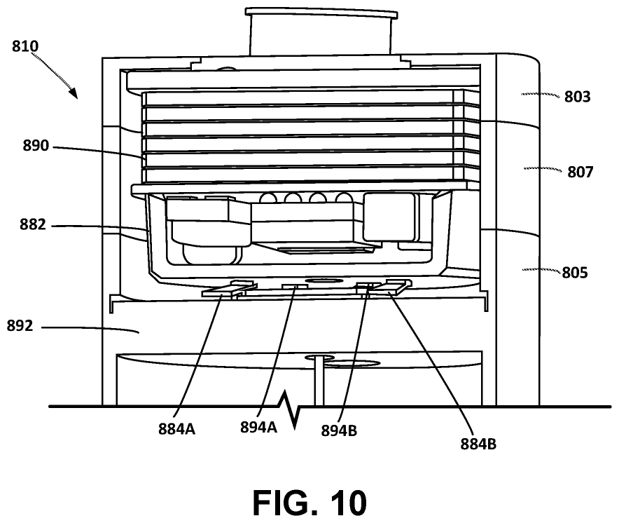

FIG. 10 is a cross-sectional diagram illustrating another example antenna 810 and another example interconnect device 882 of an IMD in accordance with techniques described in this disclosure. As illustrated in FIG. 10, antenna 810 may include a non-conductive component 807 between adjacent metal portions 803 and 805 on opposite longitudinal sides of non-conductive component 807, similar to metal portions 203,303 and 205,305 and non-conductive components 207,307 of FIGS. 2A-2C and FIGS. 3A and 3B. The IMD also includes an interconnect device 882, circuitry 890, and (although not illustrated in FIG. 10) shorts, similar to interconnect device 682, circuitry 690, and shorts 686 described above with respect to FIGS. 8A and 8B. Antenna 810 does not necessarily include one or more flex circuits configured to provide electrical contacts to metal portions 803 and 805.

FIG. 10 also illustrates elastically-deformable connectors 894A and 894B (collectively, “connectors 894”) for redundantly connecting a battery or other power source of the IMD to circuitry 890 via interconnect device 882. In the example of FIG. 10, rather than connectors that flexibly make electrical contact with a metal portion, e.g., connectors 784 with metal portion 705 as described with respect to FIGS. 9A and 9B, the IMD includes connectors 884A and 884B (collectively, “connectors 884”) that flexibly establish electrical contact between an electrical common portion of the IMD housing, in this case the battery case 892, and the RF signal source via interconnect device 882. For ease of assembly, connectors 884 may be fixedly coupled to either (but not both) of interconnect device 882 and case 892. Connectors 884 may be configured to be elastically deformable, e.g., a distance between interconnect device 882 and case 892 decreases, to flexibly maintain the electrical contact. Connectors 884 may selectively electrically connect circuitry 890 to the battery case (ground) for purposes of powering the IMD, and also for connecting the RF signal to the case to excite antenna 810.

The techniques described in this disclosure may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The following examples are illustrative of the techniques described herein.

Example 1: An implantable medical device (IMD) includes a housing; a communication circuit within the housing; and an antenna includes a non-conductive component forming part of the housing; a first metal portion of the housing adjacent to the non-conductive component on a first side of the non-conductive component; a second metal portion of the housing adjacent to the non-conductive component on a second side of the non-conductive component; and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Example 2: The IMD of example 1, wherein the non-conductive component is formed of at least one of sapphire or ceramic.

Example 3: The IMD of example 1 or 2, wherein the non-conductive component is at least partially transparent to RF waves.

Example 4: The IMD of any one or more of examples 1-3, wherein the non-conductive component comprises a polygonal or elliptical outer boundary.

Example 5: The IMD of any one or more of examples 1-3, wherein the non-conductive component is ring-shaped.

Example 6: The IMD of example 5, wherein the non-conductive component comprises a continuous ring or a plurality of segments arranged as a ring.

Example 7: The IMD of example 5 or 6, wherein the first and second metal portions are ring-shaped and have a common longitudinal axis with each other and the non-conductive component.

Example 8: The IMD of any one or more of examples 5-7, wherein the plurality of switchable contacts are distributed circumferentially around the IMD.

Example 9: The IMD of any one or more of examples 1-8, wherein the plurality of switchable contacts comprises a first subset of the plurality of switchable contacts on the first side of the non-conductive component and a second subset of the plurality of switchable contacts on the second side of the non-conductive component.

Example 10: The IMD of example 9, wherein the first subset of the plurality of switchable contacts is formed on the first metal portion and the second subset of the plurality of switchable contacts is formed on the second metal portion.

Example 11: The IMD of example 8 and example 9 or 10, wherein each contact of the first subset of the plurality of switchable contacts forms a pair with a respective contact of the second subset of the plurality of switchable contacts, wherein each pair is located at a respective position of a circumference of the IMD.

Example 12: The IMD of example 11, wherein two of the pairs of contacts are spaced 180 degrees from each other.

Example 13: The IMD of any one or more of examples 1-12, wherein the RF wireless communications comprise Bluetooth Low Energy signals.

Example 14: The IMD of any one or more of examples 1-13, further comprising one or more additional contacts that are fixed in a shorted configuration.

Example 15: The IMD of example 14, wherein the one or more additional contacts that are fixed in a shorted configuration to short the first metal portion to the second metal portion.

Example 16: The IMD of any one or more of examples 1-15, wherein each excitation mode of the plurality of excitation modes is associated with a respective radiation pattern of a plurality of radiation patterns that are complementary to each other.

Example 17: The IMD of any one or more of examples 1-16, further comprising processing circuitry within the housing, the processing circuitry configured to configure the plurality of switchable contacts.

Example 18: The IMD of example 17, wherein the processing circuitry is configured to: determine a metric of the wireless communications with the external device for at least two excitation modes of the plurality of excitation modes; select an excitation mode from the at least two excitation modes based on the metrics of wireless communication for the at least two excitation modes; and configure the plurality of switchable contacts to provide the selected excitation mode.

Example 19: The IMD of any one or more of examples 1-18, further comprising one or more flex circuits within the housing, the one or more flex circuits configured to conform to the inner surface of the antenna, wherein the one or more flex circuits comprises the plurality of switchable contacts.

Example 20: The IMD of any one or more of examples 1-19, further comprising an interconnect device configured to connect the plurality of contacts to the communication circuit.

Example 21: The IMD of any one or more of examples 1-20, wherein the IMD is configured such that the antenna is in contact with at least one of tissue or fluid of the patient when the IMD is implanted within the patient.

Example 22: The IMD of any one or more of examples 1-21, further comprising one or more electrodes integrated into the housing.

Example 23: The IMD of example 22, wherein the IMD comprises a pacemaker includes a sensing circuit within the housing, the sensing circuit configured to sense a cardiac electrogram via the one or more electrodes; and a therapy delivery circuit within the housing, the therapy delivery circuit configured to deliver pacing pulses via the one or more electrodes, wherein the housing is configured for implantation within a heart chamber of the patient.

Example 24: A pacemaker includes a housing configured for implantation within a heart chamber of a patient; a plurality of electrodes integrated into the housing; a sensing circuit within the housing, the sensing circuit configured to sense a cardiac electrogram via the one or more electrodes; a therapy delivery circuit within the housing, the therapy delivery circuit configured to deliver pacing pulses via the one or more electrodes; a communication circuit within the housing; and an antenna includes a non-conductive component forming part of the housing; a first metal portion of the housing adjacent to the non-conductive component on a first side of the non-conductive component; a second metal portion of the housing adjacent to the non-conductive component on a second side of the non-conductive component; and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Example 25: The pacemaker of example 24, wherein the non-conductive component and the first and second metal portions are ring-shaped, the first metal portion and the second metal portion having a common longitudinal axis with each other and the non-conductive component, and wherein the plurality of contacts comprises a first subset of the plurality of contacts formed on the first metal portion on the first side of the non-conductive component and a second subset of the plurality of contacts formed on the second portion on the second side of the non-conductive component.

Example 26: A method includes configuring, by processing circuitry within a housing of an implantable medical device (IMD), a plurality of switchable contacts of an antenna of the IMD according to a selected excitation mode of a plurality of excitation modes; exciting, by a communication circuit within the housing of the IMD, the antenna via the plurality of switchable contacts includes a non-conductive component forming part of the housing; a first metal portion of the housing adjacent to the non-conductive component on a first side of the non-conductive component; and a second metal portion of the housing adjacent to the non-conductive component on a second side of the non-conductive component; and communicating, by the IMD and via radiofrequency (RF) wireless communications, with an external device using the excited antenna.

Example 27: The method of example 26, wherein the non-conductive component is formed of at least one of sapphire or ceramic.

Example 28: The method of example 26 or 27, wherein the non-conductive component is at least partially transparent to RF waves.

Example 29: The method of any one or more of examples 26-28, wherein the non-conductive component comprises a polygonal or elliptical outer boundary.

Example 30: The method of any one or more of examples 26-28, wherein the non-conductive component is ring-shaped.

Example 31: The method of example 30, wherein the non-conductive component comprises a continuous ring or a plurality of segments arranged as a ring.

Example 32: The method of example 30 or 31, wherein the first and second metal portions are ring-shaped and have a common longitudinal axis with each other and the non-conductive component.

Example 33: The method of any one or more of examples 30-32, wherein the plurality of switchable contacts are distributed circumferentially around the IMD.

Example 34: The method of any one or more of examples 26-33, wherein the plurality of switchable contacts comprises a first subset of the plurality of switchable contacts on the first side of the non-conductive component and a second subset of the plurality of switchable contacts on the second side of the non-conductive component.

Example 35: The method of example 34, wherein the first subset of the plurality of switchable contacts is formed on the first metal portion and the second subset of the plurality of switchable contacts is formed on the second metal portion.

Example 36: The method of example 33 and example 34 or 35, wherein each contact of the first subset of the plurality of switchable contacts forms a pair with a respective contact of the second subset of the plurality of switchable contacts, wherein each pair is located at a respective position of a circumference of the IMD.

Example 37: The method of example 36, wherein two of the pairs of contacts are spaced 180 degrees from each other.

Example 38: The method of any one or more of examples 26-37, wherein communicating via RF wireless communications comprises communicating via Bluetooth Low Energy signals.

Example 39: The method of any one or more of examples 26-38, wherein the IMD comprises one or more additional contacts that are fixed in a shorted configuration to short the first metal portion to the second metal portion.

Example 40: The method of any one or more of examples 26-39, wherein each excitation mode of the plurality of excitation modes is associated with a respective radiation pattern of a plurality of radiation patterns that are complementary to each other, the method further includes determining a metric of the wireless communications with the external device for at least two excitation modes of the plurality of excitation modes; selecting an excitation mode from the at least two excitation modes based on the metrics of wireless communication for the at least two excitation modes; and configuring the plurality of switchable contacts to provide the selected excitation mode.

Example 41: The method of any one or more of examples 26-40, wherein the IMD is implanted within the patient with the antenna in contact with at least one of tissue or fluid of the patient.

Example 42: The method of any one or more of examples 26-41, wherein the IMD is implanted within a heart chamber, the method further includes sensing a cardiac electrogram by the IMD; and delivering pacing pulses by the IMD.

Example 43: An implantable medical device (IMD) includes a housing; a communication circuit within the housing; and an antenna includes a ring-shaped non-conductive component forming part of the housing; a first ring-shaped metal portion of the housing adjacent to the non-conductive component on a first side of the non-conductive component; a second ring-shaped metal portion of the housing adjacent to the non-conductive component on a second side of the non-conductive component, wherein the first and second ring-shaped metal portions have a common longitudinal axis with each other and the ring-shaped non-conductive component; and a plurality of switchable contacts distributed circumferentially around the IMD and configured to connect the first and second ring-shaped metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Example 44: The IMD of example 43, wherein the non-conductive component is formed of at least one of sapphire or ceramic.

Example 45: The IMD of example 43 or 44, wherein the non-conductive component is at least partially transparent to RF waves.

Example 46: The IMD of any one or more of examples 43-45, wherein the non-conductive component comprises a continuous ring or a plurality of segments arranged as a ring.

Example 47: The IMD of any one or more of examples 43-46, wherein the plurality of switchable contacts comprises a first subset of the plurality of switchable contacts on the first side of the non-conductive component and a second subset of the plurality of switchable contacts on the second side of the non-conductive component.

Example 48: The IMD of example 47, wherein the first subset of the plurality of switchable contacts is formed on the first metal portion and the second subset of the plurality of switchable contacts is formed on the second metal portion.

Example 49: The IMD of example 47 or 48, wherein each contact of the first subset of the plurality of switchable contacts forms a pair with a respective contact of the second subset of the plurality of switchable contacts, wherein each pair is located at a respective position of a circumference of the IMD.

Example 50: The IMD of example 49, wherein two of the pairs of contacts are spaced 180 degrees from each other.

Example 51: The IMD of any one or more of examples 43-50, wherein the RF wireless communications comprise Bluetooth Low Energy signals.

Example 52: The IMD of any one or more of examples 43-51, further comprising one or more additional contacts that are fixed in a shorted configuration.

Example 53: The IMD of example 52, wherein the one or more additional contacts that are fixed in a shorted configuration to short the first metal portion to the second metal portion.

Example 54: The IMD of any one or more of examples 43-53, wherein each excitation mode of the plurality of excitation modes is associated with a respective radiation pattern of a plurality of radiation patterns that are complementary to each other.

Example 55: The IMD of any one or more of examples 43-54, further comprising processing circuitry within the housing, the processing circuitry configured to configure the plurality of switchable contacts.

Example 56: The IMD of example 55, wherein the processing circuitry is configured to: determine a metric of the wireless communications with the external device for at least two excitation modes of the plurality of excitation modes; select an excitation mode from the at least two excitation modes based on the metrics of wireless communication for the at least two excitation modes; and configure the plurality of switchable contacts to provide the selected excitation mode.

Example 57: The IMD of any one or more of examples 43-56, further comprising one or more flex circuits within the housing, the one or more flex circuits configured to conform to the inner surface of the antenna, wherein the one or more flex circuits comprises the plurality of switchable contacts.

Example 58: The IMD of any one or more of examples 43-57, further comprising an interconnect device configured to connect the plurality of contacts to the communication circuit.

Example 59: The IMD of any one or more of examples 43-58, wherein the IMD is configured such that the antenna is in contact with at least one of tissue or fluid of the patient when the IMD is implanted within the patient.

Example 60: The IMD of any one or more of examples 43-59, further comprising one or more electrodes integrated into the housing.

Example 61: The IMD of example 60, wherein the IMD comprises a pacemaker includes a sensing circuit within the housing, the sensing circuit configured to sense a cardiac electrogram via the one or more electrodes; and a therapy delivery circuit within the housing, the therapy delivery circuit configured to deliver pacing pulses via the one or more electrodes, wherein the housing is configured for implantation within a heart chamber of the patient.

Example 1A: An implantable medical device (IMD) includes a housing; a communication circuit within the housing; and an antenna includes a non-conductive component forming part of the housing; a first metal portion of the housing adjacent to the non-conductive component on a first side of the non-conductive component; a second metal portion of the housing adjacent to the non-conductive component on a second side of the non-conductive component; and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Example 2A: The IMD of example 1A, wherein the non-conductive component is formed of at least one type of ceramic material.

Example 3A: The IMD of any of examples 1A or 2A, wherein the non-conductive component is at least partially transparent to RF waves.

Example 4A: The IMD of any one or more of examples 1A-3A, wherein the non-conductive component comprises a polygonal or elliptical outer boundary.

Example 5A: The IMD of any one or more of examples 1A-3A, wherein the non-conductive component is ring-shaped.

Example 6A: The IMD of example 5A, wherein the non-conductive component comprises a continuous ring or a plurality of segments arranged as a ring.

Example 7A: The IMD of any of examples SA or 6A, wherein the first and second metal portions are ring-shaped and have a common longitudinal axis with each other and the non-conductive component.

Example 8A: The IMD of any one or more of examples 5A-7A, wherein the plurality of switchable contacts are distributed circumferentially around the IMD.

Example 9A: The IMD of any one or more of examples 1A-8A, wherein the plurality of switchable contacts comprises a first subset of the plurality of switchable contacts on the first side of the non-conductive component and a second subset of the plurality of switchable contacts on the second side of the non-conductive component.

Example 10A: The IMD of example 9A, wherein the first subset of the plurality of switchable contacts is formed on the first metal portion and the second subset of the plurality of switchable contacts is formed on the second metal portion.

Example 11A: The IMD of any one or more of examples 1A-10A, further comprising one or more additional contacts that are fixed in a shorted configuration, wherein the one or more additional contacts that are fixed in a shorted configuration to short the first metal portion to the second metal portion.

Example 12A: The IMD of any one or more of examples 1A-11A, wherein each excitation mode of the plurality of excitation modes is associated with a respective radiation pattern of a plurality of radiation patterns that are complementary to each other.

Example 13A: The IMD of any one or more of examples 1A-12A, further comprising processing circuitry within the housing, the processing circuitry configured to configure the plurality of switchable contacts.

Example 14A: The IMD of example 13A, wherein the processing circuitry is configured to: determine a metric of the wireless communications with the external device for at least two excitation modes of the plurality of excitation modes; select an excitation mode from the at least two excitation modes based on the metrics of wireless communication for the at least two excitation modes; and configure the plurality of switchable contacts to provide the selected excitation mode,

Example 15A: The IMD of any one or more of examples 1A-14A, further comprising one or more flex circuits within the housing, the one or more flex circuits configured to conform to the inner surface of the antenna, wherein the one or more flex circuits comprises the plurality of switchable contacts.

Example 16A: The IMD of any one or more of examples 1A-15A, further comprising an interconnect device configured to connect the plurality of contacts to the communication circuit.

Example 17A: The IMD of example 16A, further comprising one or more elastically deformable connectors configured to connect the interconnect device to one of the first metal portion or the second metal portion.

Example 18A: The IMD of any one or more of examples 1A-17A, further includes a sensing circuit within the housing, the sensing circuit configured to sense a cardiac electrogram via the one or more electrodes; and a therapy delivery circuit within the housing. the therapy delivery circuit configured to deliver pacing pulses via the one or more electrodes, wherein the housing is configured for implantation within a heart chamber of the patient.

Example 19A: A pacemaker includes a housing configured for implantation within a heart chamber of a patient; a plurality of electrodes integrated into the housing; a sensing circuit within the housing, the sensing circuit configured to sense a cardiac electrogram via the one or more electrodes; a therapy delivery circuit within the housing, the therapy delivery circuit configured to deliver pacing pulses via the one or more electrodes; a communication circuit within the housing; and an antenna includes a non-conductive component forming part of the housing; a first metal portion of the housing adjacent to the non-conductive component on a first side of the non-conductive component; a second metal portion of the housing adjacent to the non-conductive component on a second side of the non-conductive component; and a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Example 20A: An implantable medical device (IMD) includes a housing; a communication circuit within the housing; and an antenna includes a ring-shaped non-conductive component forming part of the housing; a first ring-shaped metal portion of the housing adjacent to the non-conductive component on a first side of the non-conductive component; a second ring-shaped metal portion of the housing adjacent to the non-conductive component on a second side of the non-conductive component, wherein the first and second ring-shaped metal portions have a common longitudinal axis with each other and the ring-shaped non-conductive component; and a plurality of switchable contacts distributed circumferentially around the IMD and configured to connect the first and second ring-shaped metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

Various examples have been described. These and other examples are within the scope of the following claims.

1. An implantable medical device (IMD) comprising:
a housing;
a communication circuit within the housing; and
an antenna comprising:
a non-conductive component forming part of the housing;
a first metal portion of the housing adjacent to the non-conductive component on a first side of the non-conductive component;
a second metal portion of the housing adjacent to the non-conductive component on a second side of the non-conductive component; and
a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

2. The IMD of claim 1, wherein the non-conductive component is formed of at least one type of ceramic material.

3. The IMD of claim 1, wherein the non-conductive component is at least partially transparent to RF waves.

4. The IMD of claim 1, wherein the non-conductive component comprises a polygonal or elliptical outer boundary.

5. The IMD of claim 1, wherein the non-conductive component is ring-shaped.

6. The IMD of claim 5, wherein the non-conductive component comprises a continuous ring or a plurality of segments arranged as a ring.

7. The IMD of claim 5, wherein the first and second metal portions are ring-shaped and have a common longitudinal axis with each other and the non-conductive component.

8. The IMD of claim 5, wherein the plurality of switchable contacts are distributed circumferentially around the IMD.

9. The IMD of claim 1, wherein the plurality of switchable contacts comprises a first subset of the plurality of switchable contacts on the first side of the non-conductive component and a second subset of the plurality of switchable contacts on the second side of the non-conductive component.

10. The IMD of claim 9, wherein the first subset of the plurality of switchable contacts is formed on the first metal portion and the second subset of the plurality of switchable contacts is formed on the second metal portion.

11. The IMD of claim 1, further comprising one or more additional contacts that are fixed in a shorted configuration,
wherein the one or more additional contacts that are fixed in a shorted configuration to short the first metal portion to the second metal portion.

12. The IMD of claim 1, wherein each excitation mode of the plurality of excitation modes is associated with a respective radiation pattern of a plurality of radiation patterns that are complementary to each other.

13. The IMD of claim 1, further comprising processing circuitry within the housing, the processing circuitry configured to configure the plurality of switchable contacts.

14. The IMD of claim 13, wherein the processing circuitry is configured to:
determine a metric of the wireless communications with the external device for at least two excitation modes of the plurality of excitation modes;
select an excitation mode from the at least two excitation modes based on the metrics of wireless communication for the at least two excitation modes; and
configure the plurality of switchable contacts to provide the selected excitation mode.

15. The IMD of claim 1, further comprising one or more flex circuits within the housing, the one or more flex circuits configured to conform to the inner surface of the antenna, wherein the one or more flex circuits comprises the plurality of switchable contacts.

16. The IMD of claim 1, further comprising an interconnect device configured to connect the plurality of contacts to the communication circuit.

17. The IMD of claim 16, further comprising one or more elastically deformable connectors configured to connect the interconnect device to one of the first metal portion or the second metal portion.

18. The IMD of claim 1, further comprising one or more electrodes integrated into the housing,
wherein the IMD comprises a pacemaker comprising:
a sensing circuit within the housing, the sensing circuit configured to sense a cardiac electrogram via the one or more electrodes; and
a therapy delivery circuit within the housing, the therapy delivery circuit configured to deliver pacing pulses via the one or more electrodes,
wherein the housing is configured for implantation within a heart chamber of the patient.

19. A pacemaker comprising:
a housing configured for implantation within a heart chamber of a patient;
a plurality of electrodes integrated into the housing;
a sensing circuit within the housing, the sensing circuit configured to sense a cardiac electrogram via the one or more electrodes;
a therapy delivery circuit within the housing, the therapy delivery circuit configured to deliver pacing pulses via the one or more electrodes;
a communication circuit within the housing; and
an antenna comprising:
a non-conductive component forming part of the housing;
a first metal portion of the housing adjacent to the non-conductive component on a first side of the non-conductive component;
a second metal portion of the housing adjacent to the non-conductive component on a second side of the non-conductive component; and
a plurality of switchable contacts configured to connect the first and second metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

20. An implantable medical device (IMD) comprising:
a housing;
a communication circuit within the housing; and
an antenna comprising:
a ring-shaped non-conductive component forming part of the housing;
a first ring-shaped metal portion of the housing adjacent to the non-conductive component on a first side of the non-conductive component;
a second ring-shaped metal portion of the housing adjacent to the non-conductive component on a second side of the non-conductive component, wherein the first and second ring-shaped metal portions have a common longitudinal axis with each other and the ring-shaped non-conductive component; and
a plurality of switchable contacts distributed circumferentially around the IMD and configured to connect the first and second ring-shaped metal portions to the communication circuit, wherein the plurality of switchable contacts are configurable according to a selected excitation mode of a plurality of excitation modes to connect the communication circuit to the first and second metal portions via a selected one or more of the plurality of contacts to excite the antenna to provide radiofrequency (RF) wireless communications between the IMD and an external device.

21. A method comprising:
configuring, by processing circuitry within a housing of an implantable medical device (IMD), a plurality of switchable contacts of an antenna of the IMD according to a selected excitation mode of a plurality of excitation modes;
exciting, by a communication circuit within the housing of the IMD, the antenna via the plurality of switchable contacts, the antenna comprising:
a non-conductive component forming part of the housing;
a first metal portion of the housing adjacent to the non-conductive component on a first side of the non-conductive component; and
a second metal portion of the housing adjacent to the non-conductive component on a second side of the non-conductive component; and
communicating, by the IMD and via radiofrequency (RF) wireless communications, with an external device using the excited antenna.
